(19) United States
(12) Patent Application Publication
(10) Pub. No.: US 2018/0064415 A1
(43) Pub. Date: Mar. 8, 2018

(54) Acoustic ablation assisted intra-cardiac echocardiography catheter

(71) Applicant: Siemens Medical Solutions USA, Inc., Malvern, PA (US)

(72) Inventors: Liang Zhai, Castro Valley, CA (US); Alejandro Rendon Sanchez, Santa Clara, CA (US); Lex J. Garbini, El Granada, CA (US)

(21) Appl. No.: 15/258,595

(22) Filed: Sep. 7, 2016

(51) Int. Cl. A61B 8/08; A61B 8/12; A61B 8/00; A61N 7/02

(52) U.S. Cl. CPC A61B 8/0883; A61B 8/12; A61B 2018/00351; A61N 7/022; A61B 8/0841; A61B 8/445

(57) ABSTRACT

By combining acoustic ablation and ultrasound imaging on a catheter with needle guidance, a transseptal puncture needle may be guided using real-time imaging, and puncture by the guided needle is aided by acoustic ablation. Acoustic ablation may be controlled spatially and/or for dosage and may not require contact with the tissue.

### BACKGROUND

The present embodiments relate to medical ultrasound imaging catheters. A patient is scanned using an acoustic array of a catheter in the patient, providing real-time images from within the patient.

These images may be used for transseptal puncture. Gaining access to the left side of the heart by transseptal puncture is often required for percutanous structural heart or electrophyiology procedures, such as implanting a left atrial appendage occlusive device, repairing a percutaneous valve, or a valvuloplasty catheter ablation to treat atrial fibrillation. Although it's possible to reach the left ventricle (LV) and left atrium (LA) by manipulating a catheter, the procedure is complicated and sometimes risky for the patients. The commonly used method is transseptal puncture, which permits a direct route to the LA and LV via the intra-atrial septum and systemic venous system. However, when the septum has increased thickness or becomes fibrotic, it may be difficult to penetrate the thickened or scarred septa with the needle. In over 1% of cases, a mechanical needle may fail to puncture the septum. The needle may slip away or bend due to the increased resistance, which increases the risk of injury that may lead to tamponade. Increased force may be applied to penetrate the intra atrial septum, but this increases the risk of cardiac tamponade, pericardial effusion, aortic root needle puncture, or right or left atrial wall needle puncture. As a consecuse the patient may develop pleuritic chest pain, stroke/transient ischaemic attack, transient ST elevation of inferior leads, or persistence of atrial septal defect.

Radiofrequency (RF) energy may be used to reduce the force required to puncture the septum. An integrated RF electrode creates a lesion at the punctuation point before advancing the needle through the septum. The RF transseptal needle may be 7.2 times more likely to cross a challenging septum with a minimal risk of tamponade and may reduce the procedure time and fluoroscopy usage by 40%. However, positioning the electrode against a specific location on tissue for ablation may be difficult in the moving heart, the ablation may be difficult to control using RF, and imaging other than fluoroscopy is not provided for real-time assessment.

### BRIEF SUMMARY

By way of introduction, the preferred embodiments described below include methods, systems, and catheters for transseptal puncture. By combining acoustic ablation and ultrasound imaging on a catheter with needle guidance, the needle may be guided using real-time imaging, and puncture by the guided needle is aided by acoustic ablation. Acoustic ablation may be controlled spatially and/or for dosage and may not require contact with the tissue.

In a first aspect, a medical ultrasound system is provided for transseptal puncture. An ultrasound transducer array is on the intra-cardiac echocardiography catheter. A needle guide connects with the intra-cardiac echocardiography catheter. The needle guide is configured to guide a needle to a field of view of the ultrasound transducer array. An acoustic ablation transducer on or in the intra-cardiac echocardiography catheter is configured to direct acoustic energy within the field of view of the ultrasound transducer array.

In a second aspect, a method is provided for transseptal puncture. An image of a septum of a patient is ultrasonically generated with a transducer in a catheter. A puncture location of the septum shown in the image is acoustically ablated. A needle is guided to the puncture location with a guide of the catheter. The septum is punctured with the needle at the puncture location after the acoustic ablation.

In a third aspect, an intra-cardiac echocardiography catheter includes an ablation transducer configured to acoustically ablate tissue at a focal location, and a guide configured to guide a needle to the tissue at the focal location.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a medical ultrasound system for transseptal puncture;

FIG. 2 illustrates one embodiment of an arrangement of imaging and ablation arrays for a catheter;

FIG. 3 is a side view of one embodiment of intra-cardiac echocardiography catheter with included needle guide and ablation transducer;

FIG. 4 is a cross-sectional view of another embodiment of intra-cardiac echocardiography catheter with included needle guide and ablation transducer; and

FIG. 5 is a flow chart diagram of one embodiment of a method for transseptal puncture with a combined acoustic ablation and ultrasound imaging catheter.

### DETAILED DESCRIPTION OF THE DRAWINGS AND PRESENTLY PREFERRED EMBODIMENTS

An acoustic ablation assisted intra-cardiac echocardiograpy (ICE) catheter combines ultrasound ablation and ultrasound image guidance in a single catheter to improve transseptal puncture. The puncture needle and/or a guide for the needle may also be integrated. The integrated acoustic monitoring and ablation may help eliminate the need for multiple catheter devices to perform transseptal puncture. The ablation is accomplished using focused ultrasound directed to the desired location of the transseptal puncture. Acoustic ablation assists needle-based puncture. Compared to RF ablation, a controlled dose of acoustic power may be easily focused and steered inside or outside of the cardiac tissue without need of direct contact. The shape of the lesion and/or thermal dosage may be easily controlled. The acoustic array may also be used to image the internal structures and guide the procedure.

FIG. 1 shows a system for medical ultrasound system for transseptal puncture. The medical ultrasound system assists puncture by providing needle guidance as well as acoustic ablation. The tissue may be denatured or otherwise rendered more easily punctured by generation of heat due to focused ultrasound. This ablation and the needle are guided using ultrasound imaging.

The medical ultrasound system includes the intra-cardiac echocardiography catheter 12 with an array 14 of elements 16 for imaging and an ablation transducer 18 for ablation within the housing 20, a beamformer 24, an image processor 26, and a display 28. Additional, different, or fewer components may be provided. For example, the system includes the array 14 in a catheter 12 without the beamformer 24, image processor 26, and/or display 28. These imaging electronics may be in a separate ultrasound imaging system. The catheter 12 releasably connects with the imaging system. As another example, the imaging array 14 and ablation transducer 18 may be combined into one device, such as the array 14 of elements 16 being usable for both imaging and ablation in a time interleaved manner.

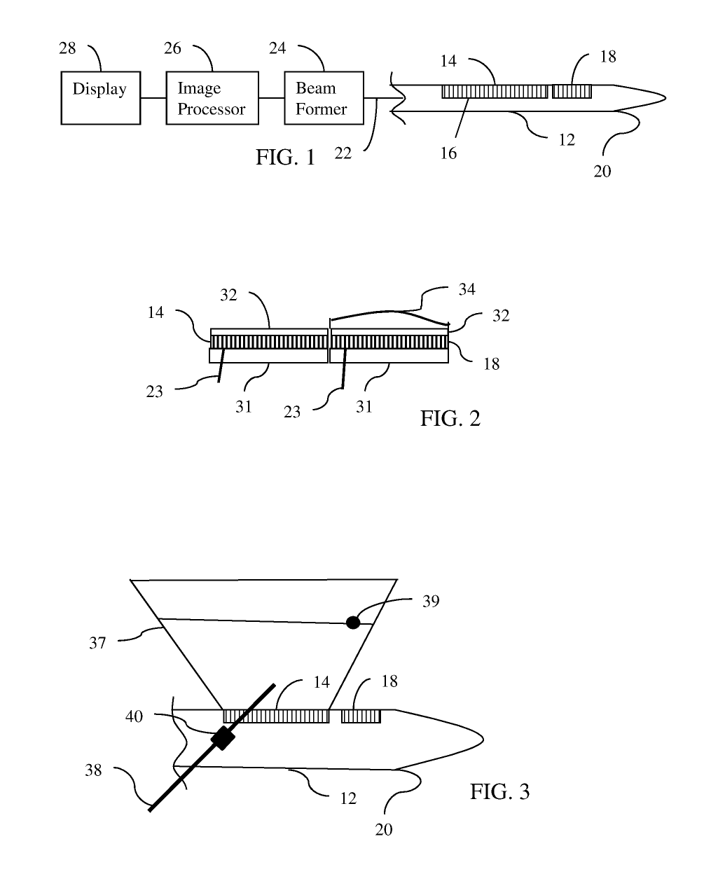

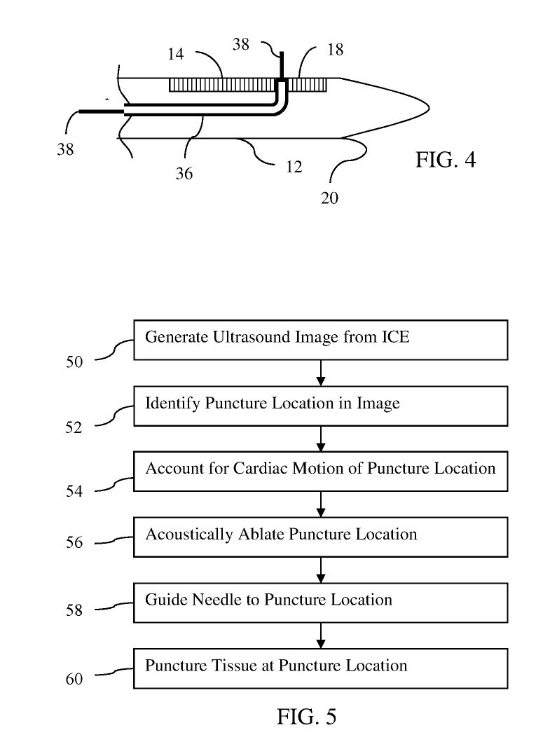

The intra-cardiac echocardiography catheter 12 includes the imaging array 14 and the ablation transducer 18 in a housing 20 with electrical conductors 22. Additional, different, or fewer components may be provided, such as steering wires, radio opaque markers, or ports. The catheter 12 may or may not include one or more needle guides 36, 40 (see FIGS. 3 and 4).

The housing 20 is PEBAX, nylon, polymer, or other flexible material. The housing 20 is formed around the array 14 and ablation transducer 18. In other embodiments, the housing 20 includes one or more windows or openings through which the array 14 and/or ablation transducer 18 are exposed. The housing 20 is configured for insertion into a circulatory system of a patient. For example, a distal tip of the catheter 20 includes a more flexible portion covered by the housing 20 for moving through the circulatory system. Steering wires connected to the housing 20 or parts within the housing 20 are configured to guide the housing 20 within the circulatory system.

The array 14 is connected with or positioned in the catheter 12. An acoustic window, lens, or housing 20 covers the array 14 to allow acoustic scanning from an emitting face of the array 14 from within the catheter.

Referring to FIG. 2, the array 14 has a plurality of elements 16, backing block 31, electrodes, and a matching layer 32. Additional, different, or fewer components may be provided. For example, two, three, or more matching layers 32 are used. The backing block material absorbs and/or reflects acoustic energy. The matching layers 32 provide a more gradual transition between acoustic impedance, minimizing reflection from the boundary between the transducer and the patient. The electrodes interact with the elements 16 to transduce between acoustic and electrical energy. The variation of potential or distance between electrodes across an element 16 causes electrical signal or acoustic energy generation, respectively.

In one embodiment, a flex circuit 23 resides between the backing block 31 and the piezoelectric of the array 14. The flex circuit 23 bends around the side of the backing block 31 and is folded (in an accordion fashion) behind the backing block 31. Within the flex connection bundle (accordion), the flex circuit 23 is connected to a bundle of conductors 22 that carry the signals between the beamformer 24 and the array 14. Other arrangements of the flex circuit 23 and/or conductors for electrically connecting with each element 16 may be provided. A flex circuit, metal sheet, or electrode connects on an opposite side of each element 16 from the flex circuit 23 to act as a grounding plane.

Referring again to FIG. 1, the elements 16 contain piezoelectric material. Solid or composite piezoelectric materials may be used. Each element is a rectangular solid, cube, or six sided, but other surfaces may be provided. For example, the emitting face of one or more elements 16 is concave or convex for elevation focusing or frequency based directivity. Alternatively, a microelectromechanical device, such as a flexible membrane over a cavity, is used. Any now known or later developed ultrasound transducer may be used.

Any number of elements 16 may be provided, such as 64 elements. 128 or other number of elements 16 may allow for larger apertures and/or a greater number of apertures. The elements 16 are adjacent to each other, such as having wavelength or less spacing between the centers of adjacent elements 16. For example, the elements 16 have half wavelength spacing with kerfs acoustically separating each element 24. Sparse arrays 12 with greater spacing between elements 16 may be used.

The elements 16 are positioned along an azimuth axis. For a one-dimensional array 14, the elements 16 are in a single row along the azimuth axis. The array 14 may be linear or curved linear. A curved linear array 14 has ends or a middle that extend towards or away in range from the azimuth axis, but the elements 16 are still positioned along the azimuth dimension. Due to the curve, some elements 16 of the array 14 are at different depths or ranges. For use in a catheter, the azimuth axis is along the longitudinal axis of the catheter 12, but may be offset from the axis or centered along the axis. The array 14 of the elements 16 is of any length, such as 7 mm, 14 mm, or 28 mm.

Multi-dimensional arrays 14 may be used. For example, two or more rows of elements 16 are adjacent to each other along the elevation dimension. 1.25, 1.5, 1.75 or 2D arrays may be provided. The spacing between elements 16 along the elevation dimension is the same or different than along the azimuth dimension, such as a 2×64 array with half wavelength spacing between all adjacent elements in azimuth. The elements are long in elevation, such as having a 3-20 wavelength elevation width, but may be half wavelength or have other spacing.

In one embodiment for volume imaging from a thin and long catheter, the array 14 twists about the longitudinal axis of the array or a longitudinal axis spaced from the center of the array. Different elements 16 or groups of elements 16 face in different directions. The change in direction along the length of the array 14 is gradual, but may have any step size. For example, the twist follows a helical pattern. By walking an aperture along the array 14, different scan planes spaced or fanned apart in elevation are defined and used for scanning. This allows scanning of a volume with the linear array.

The helical or other twist of the array 14 about any longitudinal axis is created by forming the stack and twisting the stack and/or by assembling the elements 16 in the desired relationship. In one embodiment, the transducer stack including the elements 16 is formed on or connected to a memory metal, such as Nitinol. Once cured and/or bonded with the memory metal in a flat configuration, the memory metal is forced by temperature or other energy to return to a twisted configuration. This twists the arrangement of the elements 16. Memory metal may alternatively be used to create curvature for a physical focus of the array in azimuth.

The side of the elements 16 covered by the matching layer 32, closer to the region to be scanned and/or opposite the backing block, is the emitting face of the array 14. Acoustic energy is transmitted from and received at the emitting face of the array 14. The angle of acoustic energy relative to the emitting face affects the sensitivity of the elements 16 to the energy. The elements 16 are more sensitive to the energy at normal incidence to the elements 16. Referring to FIG. 3, the array 14 is used to scan in a field of view 37 or region of desired sensitivity to any desired depth. This field of view 37 of the array 14 has any format for the corresponding scan pattern, such as Vector®, sector, or linear. The patient within the field of view 37 may be imaged using the array 14.

Referring again to FIG. 1, electrical conductors 22 connect the elements 16 of the array 14 to the receive beamformer 24. The conductors 22 are cables, coaxial cables, traces on flexible circuit material, wires, continuation of the flex circuits 23, wire jumpers, combinations thereof, or other now known or later developed conductor. One conductor 22 is provided for each element 24. Alternatively, fewer conductors 22 than elements 16 may be used, such as for switched apertures, partial beamforming, or multiplexing. The conductors 22 are separately addressable by the beamformer 24. Each element 16 may be selectively used for a given aperture and associated electronic steering. Alternatively, some elements 16 are useable with only a subset of possible apertures. One or more conductors 22 are also provided for the ablation transducer 18.

The array 14 is positioned within the catheter 12. The array 14 may fit within 10 French, 3.33 mm, 12.5 French, or other diameter catheter 12. The array 14 is at a distal end of the catheter 12, such as being within 20 mm of a tip or a beginning of a flexible tip portion. The array 14 may have any position within the catheter 12 that results in the array 14 being within the patient during use of the catheter 12 for imaging. The conductors 22 are routed through the catheter 12 to the beamformer 24. The catheter transducer array 14 is used for ultrasound imaging. The images assist in diagnosis, catheter guidance, needle guidance, ablation guidance, and/or needle puncture.

FIGS. 3 and 4 show a needle guide 36, 40 connected with the intra-cardiac echocardiography catheter 12. The needle guide 36, 40 is a structure or mechanism that positions the needle 36 relative to the field of view 37. The guide 36, 40 guides the needle along at least one spatial dimension with or without a change of angle or rotation about one or more of the dimensions. For example, the positioning provides an angle for the needle 38 relative to the catheter 12. The needle guide 36, 40 directs the needle 38 to enter the field of view 37 within 10 mm of the acoustic ablation transducer 18, the ultrasound transducer array 14, or both the acoustic ablation transducer 18 and the ultrasound transducer array 14. For example, the guide 36, 40 is positioned on or in the catheter 12 within 10 mm of one or both transducers so that the needle 38 as extended from the guide 36, 40 is within the field of view 37, whether a planar or volume field of view.

The extent of the needle 38 extending from the guide 36, 40 may be separately controlled by a motor and/or the user. The needle 38 slides through the guide 36, 40 to extend and/or retract the needle 38, such as for puncturing the septum by extending the needle 38 from the catheter 12 and then withdrawing the needle 38 from the punctured septum. The guide 40 is configured to guide the needle 38 so that a tip of the needle 38 contacts the septum and corresponding puncture location 39 within the field of view 37 and/or at a location to which the ablation transducer 18 may focus acoustic energy (i.e., focal location of a mechanically and/or electronically steered ablation transducer 18). The guide 36, 40 may be fixed or may be adjustable, such as translating and/or rotating the guide 36, 40 relative to the catheter 12.

FIG. 3 shows one embodiment of the needle guide 40. The needle guide 40 is a ring mounted or connected to the catheter, but may have other shapes with a hole through which the needle 38 extends (e.g., a sleeve fitting around the housing 20). Any mounting may be used, such as gluing, thermal bonding, molding, or integration with the catheter 12. Any material may be used, such as plastic, housing material, or metal. The hole may be formed in the housing 20 rather than being an attached structure. The housing 20 may or may not extend over the guide 40. If over, then the housing 20 forms within the guide 40 to provide the hole through which the needle 38 passes. The hole is cylindrical and of any length. Other shapes for the hole may be used.

FIG. 4 shows an alternative embodiment where the needle guide 36 forms a port on the catheter 12. The needle 38 is integrated within and/or passes through the catheter 12 to reach the patient. The needle guide 40 is a tube within the catheter 12. The tube extends along a longitudinal path or extent of the catheter 12. The tube may be angled so that the needle 38 leaves the housing 20 to extend the tip to the field of view 37. Alternatively, the tube bends as shown in FIG. 4. Any amount of bending (e.g., angle) may be used, such as less than the 90-degree bend shown in FIG. 4. The needle 38 is flexible so follows the bend in the needle guide 40 as the needle is moved towards the patient. The needle guide 40 bends the needle 30 so that the needle 38 is guided to extend into the field of view 37. The bend redirects the needle 38 from parallel to a longitudinal direction of the intra-cardiac echocardiography catheter 12 to a non-parallel angle to reach a location spaced from the intra-cardiac echocardiography catheter 12 in the field of view 37. The needle 38 exits the port in the intra-cardiac echocardiography catheter 12 at an oblique or normal angle to the longitudinal dimension. The exit is within the field of view 37 or angles the needle 38 to extend into the field of view 37. The needle 38 is forced to the puncture point 39 through the needle guide 40 inside the catheter 12. A motor, lever, screw drive, other power source, or manual force is used to push and retract the needle 38 during the procedure.

Referring to FIGS. 1-4, the acoustic ablation transducer 18 is on or in the intra-cardiac echocardiography catheter 12. The ablation transducer 18 is configured to acoustically ablate tissue at a focal location 39. The focal location 39 is fixed as a mechanical focus. Alternatively or additionally, the focal location 39 may be positioned electronically, such as using a phased array of elements as the ablation transducer 18. A single concave piston transducer, a confocal transducer, a one-dimensional array, or a two-dimensional array may be used for the ablation transducer 18. In other embodiments, the imaging array 14 is the ablation transducer 18, such as where all the elements 16 or an aperture of the array 14 is used to acoustically ablate. In yet another embodiment, elements 16 of the imaging array 14 and elements of the ablation array 18 are interspersed, such as every other, in a larger array.

FIG. 2 shows one embodiment of the transducer stack for the ablation transducer 18. A backing block 31 and any number of matching layers 32 are stacked with the transducer 18. The transducer 18 is of a same or different material, size, and/or structure as the array 14. For example, the element length for the array 14 is 2.5 mm, but the element length for the ablation transducer 18 is 7 mm. As another example, 23, 32, or a smaller number of elements are used for the ablation transducer 18 than for the imaging array 14. The transducer 18 is stacked between a grounding plane and one or more electrodes (e.g., flex circuit 23 with metallic pads or traces) for transducing between electrical and acoustic energy. The ablation transducer 18 geometry may be set to optimize the ablation. For a mechanical focus, a lens 34 is added. The lens 34 concentrates the acoustic energy at a mechanical focal point or region. The lens 34 is cylindrical, spherical, parabolic, Fresnel, or other. The lens 34 is fabricated from silicone or a material with similar acoustic focusing properties. Alternatively, the mechanical focusing is accomplished by shaping the array and/or elements of the ablation transducer 18 in a non-planar configuration. In alternative embodiments, no lens or no mechanical focus is provided.

Referring to FIG. 2, the ablation transducer 18 is positioned adjacent to or against the imaging array 14 within the catheter 12. For example, only the needle guide 36 separates the array 14 and the ablation transducer 18 (see FIG. 4). 10-20 mm, lesser, or greater separation of the closest points of the array 14 and the ablation transducer 18 may be provided. The ablation transducer 18 is positioned relative to the imaging array 14 so that the focal location 39 of the ablation transducer 18 is within or may be steered to be within the field of view 37 of the imaging array 14 and/or may be at tissue at a point of contact for the guided needle 38.

In the example of FIGS. 1, 3, and 4, the ablation transducer 18 is positioned distally on the intra-cardiac echocardiography catheter 12 to the ultrasound transducer array 14. Side-by-side, interspersed, or the imaging array 14 being distal to the ablation transducer 18 arrangements may be used. Referring to FIG. 2, the flex circuit 23 may be shared by each array (imaging array 14 and array for the ablation transducer 18), or separate flexible circuits 23 are provided.

In one embodiment, the array 14 and transducer 18 are positioned on a common baseplate. Separate baseplates may be used. Rather than just using backing block 31, the baseplate may accurately position the array 14 with respect to the ablation transducer 18. The baseplate may be fixed or stiff relative to the rest of the catheter 12 within the patient. The baseplate is stainless steel, ceramic (e.g., aluminum oxide), Nitinol, or other suitable materials. The use of Nitinol or other memory metal allows the ablation transducer 18 to be curved during the fusing process, when the temperature is heated above 140 C to flow PEBAX to form the housing 20. The assembly of the array in a planar configuration, with planar components, is advantageous from a manufacturing cost perspective. The use of the shape-memory material for the baseplate allows introduction of a mechanical focus after stacking the transducer 18.

The acoustic ablation transducer 18 is configured to direct acoustic energy within the field of view 37 of the ultrasound transducer array 14. The transmit beamformer 24 or a separate therapy transmitter generates one or more electrical waveforms with a desired intensity and/or duration for acoustic ablation. With electronic steering and/or control of the position of the catheter 12, the acoustic energy for ablation may be steered to different locations relative to the ablation transducer 18, such as to account for cardiac motion and/or to ablate different tissue locations by different amounts. The ablation transducer 18 is configured to transmit the acoustic energy to the focal location 39, such as by, on, or within the septum or other tissue for puncture. The focal location 39 is spaced from the ablation transducer 18.

In one embodiment, the ablation transducer 18 is configured to alternatively or additionally direct the acoustic energy to part of the needle 38. For example, the needle tip is positioned at the puncture location 39. The acoustic energy for ablation is focused at that puncture location 39. Some of the acoustic energy impinges on the needle 38 at various locations along the length of the needle 38. The needle 38 may be heated by the acoustic energy. Where the needle 38 includes acoustically absorbing material, such as rubber or chambers of air or other material that absorbs acoustic energy, the needle 38 increases in temperature. This increased temperature propagates to the tip in contact with the tissue. This heating of the needle 38 may act to ablate the tissue and/or reduce resistance to puncture. Alternatively, the acoustic energy does not impinge upon the needle 38 and/or the temperature of the needle 38 is not increased. Whether using needle heating and/or direct application of the acoustic energy to tissue, a thermal lesion may be created at the punctuation point in the transseptum.

The imaging function of the array 14 helps map the heart, identify the point of puncture 39, guide the needle 38, and/or guide the ablation by the ablation transducer 18. The imaging array 14 is used to generate a sequence of images for selecting the puncture point, monitoring needle placement at the puncture point, steering the ablation, monitoring ablation, and/or monitoring puncture. The images may be used to determine proper positioning of the catheter 12 within the circulatory system. The positioning of the catheter 12 may additionally or alternatively be guided by other devices such as x-ray (e.g., fluoroscopy), another ultrasound device, or external electric or magnetic field.

The needle 38 is a metal needle for puncturing the septum. Other materials may be used. The needle 38 fits through the needle guide 36, 40. The needle 38 is flexible to allow bending within the catheter 12 and/or patient. Any length may be used, such as a needle of a 10 cm connected to a guide wire or control rod for extending and retracting the needle 38 relative to the catheter 12. In one embodiment, the needle 38 is made off or includes chambers filled with acoustically absorbing material, such as a rubber, air, liquid, gas, or solid. The acoustically absorbing material is positioned at the tip, such as spaced within 5 mm of the tip, so that heating due to absorbing acoustic energy results in heating of the tip. The acoustic ablation energy generated while the tip of the needle 38 is by or against the tissue to be punctured also causes the needle 38 to increase in temperature.

Referring again to FIG. 1, the array 14 and/or the ablation transducer 18 connect to the beamformer 24. The beamformer 24 includes a plurality of channels for generating transmit waveforms and/or receiving signals. Relative delays and/or apodization focus the transmit waveforms or received signals for forming beams and setting a focal location. The beamformer 24 connects with the conductors 22 for applying waveforms for ablation with the ablation transducer 18 and/or for imaging with the array 14.

For ablation, the beamformer 24 generates one or more electrical waveforms having many (e.g., hundreds) of cycles at any intensity. The electrical waveforms are provided to the ablation transducer 18, which converts the electrical energy to acoustic energy focused at the focal location for ablation. Where an array is used, the waveforms for different elements are relatively delayed or phased to provide electronic focus at a desired location spaced from the ablation transducer 18. For example, the location for puncture of tissue is identified in an image. The focal location for the ablation transducer 18 is set to the location of the tissue using electronic steering and/or control of physical position of the transducer 18. Mechanical steering may be used to focus the acoustic energy. The acoustic energy causes the tissue to heat, forming a lesion or otherwise ablating the tissue.

For imaging, the beamformer 24 selects an aperture including one, some, or all of the elements 16 of the array 14. Different apertures may be used at different times. The aperture is formed by using the elements 16 for transmit and/or receive operations while not using other elements. The beamformer 24 is operable to scan from a plurality of apertures formed by adjacent groups of the elements 16. For scanning, the beamformer 24 electronically focuses along the azimuth direction. A plurality of scan lines using an aperture is scanned. During receive operations, the focus may vary as a function of depth (i.e., dynamic focusing). An elevation focus is provided by a lens and/or element sensitivity, or the array 14 is not focused in elevation. In alternative embodiments, the beamformer 24 connects with elevation spaced elements for at least partial electric focusing and/or steering in the elevation dimension.

The image processor 26 is a detector, filter, processor, application specific integrated circuit, field programmable gate array, digital signal processor, control processor, scan converter, three-dimensional image processor, graphics processing unit, analog circuit, digital circuit, or combinations thereof. The image processor 26 receives beamformed data and generates images on the display 28. The images are associated with a two-dimensional scan. Alternatively or additionally, the images are three-dimensional representations. Data representing a volume is acquired by scanning. The image processor 26 renders an image from the data representing the volume.

The image processor 26 or another processor is configured by hardware, firmware, and/or software to control the beamformer 24 and/or the acoustic ablation transducer 18. Since the catheter 12 and/or the tissue to be ablated moves over time due to the cardiac cycle, the beamformer 24 is controlled based on the cardiac cycle. The acoustic focal location for ablation is stabilized relative to the septum or puncture location during the cardiac cycles. For example, ultrasound imaging is used to track the septum motion. Real-time imaging, such as feature and/or speckle tracking, is used to determine the position of the puncture location at any time. The position of the location and/or the vector for rate and direction of change in the location are used to compensate for the cardiac motion. The ablation focal location is adjusted to account for the position and/or change in position, providing spatial precision and efficiency in the ablation. Motion compensation may be achieved by adjusting the focus of the acoustic array or through mechanical adjustment, such as adjusting the catheter position with motors. In an alternative embodiment, the ablation transmissions are timed to the heart cycle, such as using ECG or ultrasound measures of the heart cycle and transmitting the acoustic energy for ablation at a same phase associated with less movement (e.g., diastolic phase) and not transmitting at other phases.

In an additional or alternative approach, the needle 38 is used to lock or link the puncture location and the ablation transducer 18. By positioning the needle 38 against the tissue, the contact is used to stabilize the relative position of the ablation transducer 18 and the puncture location. As the tissue moves, this movement is transmitted to the ablation transducer 18 by the needle 38, maintaining the relative positions. Because the acoustic focus or the axial axis is always aligned with the needle 38, the contact between the needle tip and septum at least partially stabilizes the device.

For monitoring ablation, the imaging using the imaging array 14 may be used. The images may show the tissue as different, such as having a different texture or speckle pattern. Alternatively or additionally, the ultrasound imaging may be used to measure temperature of the tissue and/or tissue elasticity. The temperature or elasticity may indicate a level of ablation for guiding lesion creation. The imaging may also be used to verify application of ablation to the correct location.

FIG. 5 is a flow chart diagram of one embodiment of a method for transseptal puncture. Acoustic ablation and imaging are provided from one catheter. The acoustic ablation acts to alter tissue, making needle puncture through the tissue easier and/or more likely to succeed. The needle for puncturing is inserted separately or may be guided by the catheter.

The method is implemented by the catheter of FIG. 1, 2, 3, or 4. Alternatively, a different catheter with an imaging array and an ablation transducer is used.

Additional, different, or fewer acts may be provided. For example, acts 50, 52, and/or 54 are not performed. As another example, act 58 is not performed using the catheter and/or a guide on the catheter.

The acts are performed in the order shown or a different order. For example, act 54 is performed prior to act 52. As another example, act 58 is performed as part of act 54, so is performed prior to act 56. In yet another example, act 50 is repeated throughout the procedure, so occurs prior to, during, and/or after each act.

In act 50, an image is generated. The image is generated ultrasonically. Ultrasound transmission and reception is used to generate an image representing an area or volume of a patient. The image is generated using an imaging array in a catheter. The imaging array and catheter are inserted within a patient, such as into a heart or vessel of the patient. Once positioned or while positioning, the imaging array is used to transduce between electrical and acoustic energies to scan in a field of view of the array. The data acquired by scanning is used by an ultrasound imaging system to generate an image of the patient. A sequence of images, such as real-time imaging, may be provided. At least some of the images show the septum of the patient or other tissue to be punctured.

In act 52, a puncture location is identified. The operator or processor identify the location. For example, the user views one or more images. Using a user interface, the location is designated on one or more of the images. As another example, a processor applies image processing, such as template matching or a machine learnt classifier, to identify the location for puncture.

Since the ablation transducer has a known position relative to the imaging array for generating the image, the location identified in the image is spatially related to the ablation transducer. The focus of the ablation transducer may be shifted to the identified location. Similarly, where the needle guide is mounted to the catheter, the identification of the location in the image translates into a needle position relative to the catheter. Alternatively or additionally, the needle is imaged. Based on feedback from the imaging, the operator moves the needle, needle guide, and/or catheter to position the needle to the identified location.

In act 54, the cardiac motion is accounted for in the ablation and needle guidance. In one approach, signals representing the heart cycle (e.g., ECG signals) are used to trigger the ablation. By ablating during a same phase each cycle and not ablating during other phases, the position of the location for puncture relative to the ablation transducer stays stable during ablation. In another approach, the motion and/or position of the location for puncture over one or more heart cycles is determined. This motion or phase-based position is used to alter or steer the ablation focal location to match the changing location for puncture. In yet another approach, the needle is guided in act 58 to the puncture location. The needle pressing against the tissue stabilizes the ablation transducer relative to the tissue (e.g., motion of the tissue translates to the transducer and/or the pressure from the needle acts to reduce motion of the tissue). Other approaches may be used. More than one approach may be used, such as stabilizing with needle contact as well as phase-based triggering and/or motion correction of the focus of the ablation.

In act 56, the location to be punctured is acoustically ablated. An ablation transducer converts electrical energy from a transmitter (e.g., transmit beamformer) into acoustic energy. The acoustic energy is electronically and/or mechanically steered to the location of the septum. For example, electrical steering is used to focus the acoustic energy at the location shown in one or more of the images generated in act 50. As another example, the catheter and/or ablation transducer are moved to place a mechanical focal location at the location to be punctured.

The acoustic energy is focused at the location for ablation. The duration, intensity, and/or focal position of the acoustic energy are controlled to provide the desired dosage and dose distribution. The acoustic energy heats the tissue, causing ablation. For example, the tissue is heated at the location to denature, necrotize, or otherwise alter the tissue.

The needle, if in position adjacent to the location for puncturing the tissue, may be heated as well. By focusing the acoustic energy at the location, at least some of the acoustic energy may be absorbed by the needle or acoustically absorbing material in the needle, heating the needle. Alternatively, the focus of the acoustically ablating energy is shifted to the needle, whether spaced from or positioned against the tissue. The transmitted acoustic energy heats the needle, which is then used to puncture the tissue. Acoustic energy may be transmitted simultaneously to two focal locations (e.g., the location for puncture and a location on the needle) with multi-beam, or the focus of the acoustic energy is interleaved between the two locations.

In act 58, the needle is guided to the location for puncture. The needle is extended through the needle guide. The needle guide may be adjusted to steer the needle. Alternatively, the catheter with an attached or integrated needle guide is steered or moved to steer the needle. The needle is extended by an operators manual control, such as using a manually moved control wire or using a user interface to control a motor. The amount of force for wire movement is fixed or may be controlled.

Where the needle guide includes a bend, moving the needle through the guide bends the needle. The bend may be retained by the needle or the needle is straightened by the guide or material of the needle. For example, the needle is housed in a tube in the catheter. For guiding the needle, the needle is moved through the tube including a bend to redirect the needle to the field of view. A straight part of the tube after the bend straightens the needle, which then exits the catheter extending into the field of view of the imaging array. The needle is guided from being in a longitudinal direction along the catheter to progressing in a different direction into the field of view. The tip of the needle, while in the field of view, may be monitored with imaging from the imaging array. The imaging is used as feedback to the operator to guide the tip of the needle to the tissue location for puncture.

The guidance occurs before, during, or after the ablation of act 56. For example, the needle tip is guided to the tissue location to reduce relative movement. Once pressed against the tissue, the operator determines whether ablation is needed or desired to puncture. If needed, the ablation is activated. As another example, the tissue is ablated prior to the tip of the needle being positioned to contact the tissue.

In act 60, the tissue is punctured. The septum is punctured with the needle. The needle is forced through the tissue at the puncture location. The puncturing may occur after ablation and/or heating of the needle. The ablation may result in less force being needed to puncture the tissue with the needle. The heating of the needle may assist in ablation of the tissue, reducing force needed to puncture, and/or reducing bleeding upon puncture. Similarly, the ablation may be applied during puncture or after puncture to reduce bleeding.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A medical ultrasound system for transseptal puncture, the system comprising:
an intra-cardiac echocardiography catheter;
an ultrasound transducer array of the intra-cardiac echocardiography catheter;
a needle guide connected with the intra-cardiac echocardiography catheter, the needle guide configured to guide a needle to a field of view of the ultrasound transducer array; and
an acoustic ablation transducer on or in the intra-cardiac echocardiography catheter, the acoustic ablation transducer configured to direct acoustic energy within the field of view of the ultrasound transducer array.

2. The medical ultrasound system of claim 1 wherein the intra-cardiac echocardiography catheter comprises a housing configured for insertion into a circulatory system of a patient and steering wires configured to guide the housing within the circulatory system.

3. The medical ultrasound system of claim 1 wherein the ultrasound transducer array comprises a one-dimensional array of transducer elements for scanning a patient within the field of view, the one-dimensional array being distal end of the intra-cardiac echocardiography catheter for insertion within the patient.

4. The medical ultrasound system of claim 1 wherein the needle guide comprises a hole through which the needle passes, a structure forming the hole connected to the intra-cardiac echocardiography catheter.

5. The medical ultrasound system of claim 1 wherein the needle guide comprises a tube through the intra-cardiac echocardiography catheter.

6. The medical ultrasound system of claim 5 wherein the tube extends along a longitudinal path of the intra-cardiac echocardiography catheter, the tube bending within the intra-cardiac echocardiography catheter so that the needle is guided to extend into the field of view.

7. The medical ultrasound system of claim 1 wherein the needle guide is configured to direct the needle to enter the field of view within 10 mm of the acoustic ablation transducer, the ultrasound transducer array, or both the acoustic ablation transducer and the ultrasound transducer array.

8. The medical ultrasound system of claim 1 wherein the needle guide is configured to redirect the needle from parallel to a longitudinal direction of the intra-cardiac echocardiography catheter to a location spaced from the intra-cardiac echocardiography catheter in the field of view.

9. The medical ultrasound system of claim 1 wherein the acoustic ablation transducer comprises an ablation array positioned distally on the intra-cardiac echocardiography catheter to the ultrasound transducer array.

10. The medical ultrasound system of claim 1 wherein the acoustic ablation transducer comprises a transducer stack with a lens.

11. The medical ultrasound system of claim 1 further comprising a beamformer configured to generate electrical waveforms converted to acoustic energy for ablation by the acoustic ablation transducer, the acoustic energy focused at a location spaced from the acoustic ablation transducer, the location being identified from an image generated with the ultrasound transducer array and for the transseptal puncture by the needle.

12. The medical ultrasound system of claim 1 further comprising the needle, the needle having a tip comprising acoustic energy absorbing material such that the tip heats due to acoustic energy transmitted from the acoustic ablation transducer while the tip is positioned at a transseptal puncture location.

13. The medical ultrasound system of claim 1 further comprising a processor configured to control the acoustic ablation transducer as a function of a cardiac cycle.

14. A method for transseptal puncture, the method comprising:
ultrasonically generating an image of a septum of a patient with a transducer in a catheter;
acoustically ablating a puncture location of the septum shown in the image;
guiding a needle to the puncture location; and
puncturing the septum with the needle at the puncture location after the acoustic ablation.

15. The method of claim 14 wherein acoustically ablating comprises focusing acoustic energy at the puncture location as designated in the image.

16. The method of claim 14 wherein guiding comprises bending the needle from a longitudinal direction along the catheter to place a tip of the needle in a field of view of the transducer.

17. The method of claim 14 wherein the needle comprises acoustically absorbing material, and wherein acoustically ablating comprises transmitting acoustic energy to the acoustically absorbing material while a tip of the needle is positioned against the puncture location and transmitting the acoustic energy to the puncture location.

18. An intra-cardiac echocardiography catheter comprising:
an ablation transducer configured to acoustically ablate tissue at a focal location; and
a guide configured to guide a needle to the tissue at the focal location.

19. The intra-cardiac echocardiography catheter of claim 18 wherein the guide comprises a tube extending longitudinally along the intra-cardiac echocardiography catheter and bending such that the needle exits the intra-cardiac echocardiography catheter at an oblique angle and within a field of view of the intra-cardiac echocardiography catheter.

20. The intra-cardiac echocardiography catheter of claim 18 wherein the ablation transducer is configured to transmit acoustic energy to the tissue at the focal location while the needle is against the tissue where the needle includes acoustically absorbing material such that the needle temperature increases in response to the acoustic energy.
